(19) United States
(12) Patent Application Publication
(10) Pub. No.: US 2024/0417367 A1
(43) Pub. Date: Dec. 19, 2024

(54) FACILE DIRECT AMINATION AND ALKYLAMINATION OF CARBON NANOTUBES

(71) Applicant: BOARD OF REGENTS, THE UNIVERSITY OF TEXAS SYSTEM, Austin, TX (US)

(72) Inventors: Melissa A. WUNCH, Dallas, TX (US); Sangmin LEE, McKinney, TX (US); Duck J. YANG, Flower Mound, TX (US)

(73) Assignee: BOARD OF REGENTS, THE UNIVERSITY OF TEXAS SYSTEM, Austin, TX (US)

(21) Appl. No.: 18/701,465

(22) PCT Filed: Oct. 28, 2022
(86) PCT No.: PCT/US2022/078895

§ 371 Date: Apr. 15, 2024

Related U.S. Application Data
(60) Provisional application No. 63/273,686, filed on Oct. 29, 2021.

(51) Int. Cl. C07C 209/68; C07C 209/02; C07C 211/19; C07C 211/38

(52) U.S. Cl. CPC C07C 209/68; C07C 209/02; C07C 211/19; C07C 211/38; C07C 2603/90

(57) ABSTRACT

Disclosed herein are embodiments of methods for preparing aminated or alkylaminated CNTs wherein the aminated or alkylaminated CNTs are obtained in a reaction by reacting the CNTs with an aminating or alkylaminating reagent in a non-hazardous solvent or a non-hazardous solvent-deionized water mixture. The CNTs may be single-walled, double-walled or multi-walled CNTs. The disclosed processes for amination and alkylamination do not require treatment with concentrated acid, and with the use of solvent, the CNTs and aminating compound or alkylaminating compound are mixed thoroughly throughout the reaction.

This application claims the benefit of priority to U.S. Provisional Application No. 63/273,686, filed on Oct. 29, 2021, the entire contents of which are hereby incorporated by reference.

### FIELD

The present disclosure relates to the functionalization of Carbon Nanotubes (CNTs) for CNT-based composites and, more specifically, to methods of amination and alkylamination of CNTs.

### BACKGROUND

Carbon nanotubes (CNTs) are an allotrope of carbon, similar to graphene, which are nanometers in diameter (Dresselhaus et al., 2008). They exist as single, double, or multi-walled structures. CNTs were initially discovered in 1991 by Ijima and have been widely studied since due to their properties (Barreiro et al., 2006). These properties include high strength, thermal conductivity, and electrical conductivity. Unlike graphene, CNTs are electrically conductive along the nanotube axis. In order to improve the properties, functional groups have been introduced on the edges and surfaces of CNTs. These functional groups include: carboxyl (—COOH), alkylamine, amine, fluorine, etc.

Amination of carbon nanotubes is often performed by treatment of the CNTs with acid followed by aminating agent (Alam et al., 2014; Shanmugharaj et al., 2007; Zhang et al., 2015). The acid introduces carboxyl groups on the surface (—COOH) before functionalization with amine groups (Alam et al., 2014; Zhang et al., 2015; Raja et al., 2008). Amine functionalization is performed using different aminating agents including most commonly polyamines (Alam et al., 2014; Abdelkader Fermández et al., 2017). These compounds include polydopamine, ethylenediamine, diethylenetriamine, etc. which are often hazardous and difficult to handle (Alam et al., 2014; Abdelkader Fermández et al., 2017).

Similar to amination of CNTs, alkylamination has been performed using a wide variety of compounds. Often the alkylaminating agent has a long hydrocarbon chain consisting of 18 carbons or more. Current work also reports that prior to functionalization with alkyl groups the CNTs are treated with concentrated acid to place functional groups on the surface of the CNTs (Ferreira et al., 2017). The most common alkylaminating agent reported to produce these materials is octadecylamine (ODA), which has an 18-carbon chain with an amine group at the end. There has been reports by Basiuk et al. which outline the synthesis of alkylamination of multi-walled carbon nanotubes with ODA using gas-phase solvent-free procedure at 150-170° C. for 2 hours (Basiuk et al., 2004; Basiuk et al., 2005). They report that through treatment of CNTs with octadecylamine (ODA) and other amines in gas-phase with heat the ends of the nanotubes were functionalized with the ODA (Basiuk et al., 2004; Basiuk et al., 2005). While their work shows that it is possible to produce alkylaminated CNTs by just treating a solid mixture of ODA and other alkylamines with CNTs in a gas-phase solvent-free method with heat, it can be difficult to ensure a homogenous dispersion of the alkyl compound throughout the mix.

Therefore, there remains a need to develop methods that do not require a use of acid nor toxic chemicals.

### SUMMARY

In order to eliminate the use of these hazardous chemicals often used in the amination of CNTs, the present disclosure provides methods which introduces an environmentally friendly aminating agents and solvents for functionalization. Furthermore, the presently described methods does not require the treatment of the CNTs with concentrated acids before amination. The present disclosure describes a green approach for the functionalization of carbon nanotubes (CNTs) with amine groups (—NH₂) at 1 atmosphere using urea and propylene glycol. Unlike previous reports, these methods eliminate the use of pretreatment of the CNTs with concentrated nitric acid (HNO₃) to introduce carboxyl groups on the surface (—COOH). The present methods may also be easily scaled up due to the low cost of the raw materials (e.g., urea and propylene glycol) used for synthesis. It is also feasible to adjust the amine content within the aminated CNTs (ACNTs) through increasing or decreasing the urea or reaction time. The development of this eco-friendly method of ACNT production can provide a pathway in which amination of CNT materials can be easily performed, scaled up, and altered (i.e., reaction time or temperature). Additionally, these materials can be included as an additive in resins to help enhance their mechanical and electrical/thermal properties due to interactions between the amine groups and the resin. Upon addition in epoxy resin, amine groups can react with the epoxy groups forming covalent bonds. This can help to enhance the durability of the resin, in addition to potentially enhancing its mechanical, thermal and electrical properties as well. A similar interaction can occur upon inclusion of the aminated CNTs in a two-component (2K) urethane system, where the amine groups can react with isocyante groups present in the curing agent. This interaction can also form a covalent bond within the material, thus having the ability to enhance durability and potentially, mechanical, thermal and electrical conductivity. Aminated or alkylaminated or alkenyl aminated or tr-alkoxy functional aminated CNTs as nano-fillers can also use to prepare carbon fiber epoxy composite and fiberglass epoxy composite or carbon fiber thermoplastic composite and glassfiber thermoplastic composite, engineering plastics, resins, rubbers, paints and inks.

Disclosed herein are embodiments of methods for preparing aminated or alkylaminated CNTs wherein the aminated or alkylaminated CNTs are obtained in a reaction by reacting the CNTs with an aminating or alkylaminating reagent in a solvent or a solvent-deionized water mixture. The CNTs may be single walled, double walled or multi-walled CNTs. In some embodiments the CNTs have an average diameter in the range of 3 nm to 50 nm, In other embodiments, the CNTs comprise at least one of reduced chemically CNTs. In an embodiment, the mass percentage of carbon in the CNTs is in the range of 95.0%-99.9% where impurity is Fe, Co, Ni, oxygen or combination thereof. Fe, Co, Ni and other metal are being used for CNT synthesis as known in the art.

The aminating and/or alkylaminating or other aminating reagent may be urea, a mono-alkyl amine, a di-alkyl amine, ethylenediamine, para-phenylenediamine, diethylamine, trimethylamine, hexylamine, octylamine, dodecylamine, octadecylamine, oleylamine, and tri-alkoxy silyl propyl amine or a combination thereof. In some embodiments, the ratio of the CNT to any of the reagents listed above is in the range of 0.1 to 5.0 by weight. In some embodiments, the deionized water-solvent mixture includes a triol-based solvent comprising glycerol. The deionized water-solvent mixture includes at least one of: ethylene glycol, diethylene glycol, triethylene glycol, tetraethylene glycol, propylene glycol, any high boiling glycol, or a triol based solvent such as glycerol. Preferred solvent are non-toxic propylene glycol and glycerol. The deionized water-solvent mixture has a deionized water content in the range of 0%-30% by volume, preferably 0%-10% by volume. The solvent may be ethylene glycol, diethylene glycol, triethylene glycol, tetraethylene glycol, propylene glycol, any high boiling glycol, a triol based solvent like glycerol or a combination thereof.

The temperature of the reaction is in the range of 150-250° C., preferably at 180° C. at 1 atmospheric pressure, under reflux in air or under inert conditions. The duration of the reaction is in the range of 1-24 hours, preferably is in the range 1-10 hours, most preferably 1-5 hours.

The aminated CNT compound, as prepared by the method disclosed herein, has a mass ratio of amine functional groups (—NH₂) to mass of CNT in the range of 0.001 to 0.02. Additionally, the aminated CNT compound, as prepared by the method, typically has a mass percentage of oxygen in the range 0%<O %<0.5%.

The alkylaminated CNT compound, as prepared by the method disclosed herein, has a mass ratio of alkylamine functional groups (—Cₙ₊₁, where n:5-19) to mass of CNT is in the range of 0.005 to 0.05. Additional amines are alkenyl amine such as oleylamine and tri-alkoxy functional amine such as tri-ethoxy silyl propylamine.

In some embodiments, the aminated CNT compound, as prepared by the method disclosed herein, is included as a component in an ink, a coating, a polymer & resin, a composite, a rubber, an engineering plastic, a fiber or a polymer film wherein the coating may be a urethane coating or an epoxy coating or unsaturated polyester resin or vinyl ester resin or other coatings and wherein the engineering plastic may be Nylon 6, Nylon 66, PET (polyester), PC (polycarbonate), other polar polymers, and combinations thereof, and polyethylene and polypropylene, other non-polar polymers and combinations thereof and wherein the resin is UV curing resins and wherein composite can be glass fiber epoxy composite, carbon fiber epoxy composite, glass fiber thermoplastic composite, carbon fiber thermoplastic composite, glass fiber vinyl ester composite, carbon fiber vinyl ester composite and rubbers for EV tires, bicycle tires and motocycle tires.

In some embodiments, the alkylaminated CNT (Alk-CNT) compound, as prepared by the method disclosed herein, is included as a component in an ink, a coating, a polymer, a composite, an engineering plastic, a rubber, a fiber or a polymer film wherein the coating may be a urethane or an epoxy coating or unsaturated polyester resin or other coatings, and wherein the engineering plastic may be polypropylene, polyethylene, TPO (thermoplastic poly olefins), rubber, other non-polar plastics and combinations thereof.

Our processes for amination or alkylamination of CNTs do not require treatment with concentrated acid, and through the use of solvent, we ensure that the CNTs and alkylaminating compound are mixed thoroughly throughout the reaction.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The novel features believed characteristic of the illustrative embodiments are set forth in the appended claims. The illustrative embodiments, however, as well as a preferred mode of use, further objectives and features thereof, will best be understood by reference to the following detailed description of an illustrative embodiment of the present disclosure when read in conjunction with the accompanying drawings, wherein:

FIG. 1 is a plot of the FTIR spectra of an amine functionalized CNT using a reflux method according to an embodiment of the present invention.

FIG. 2 is a plot of XPS N1s spectra of pristine CNT and aminated CNT

FIG. 3 is a graph showing TGA analysis of CNT materials CNT and ACNT according to an embodiment of the present invention

FIG. 4 is a plot of the Instron measurements of an amine functionalized CNT in an epoxy composite

FIG. 5 is a plot of the FTIR Spectra of an alkylaminated CNT synthesized using a reflux method according to an embodiment of the present invention.

FIG. 6 is a graph showing TGA analysis of CNT materials CNT and Alk-CNT according to an embodiment of the present invention.

FIG. 7 is a picture showing dispersion stability of Alk-CNT in a non-polar organic solvent versus CNT after two weeks.

FIG. 8 is a flow diagram showing a method for direct amination of CNTs to form an ACNT according to an embodiment of the present invention.

FIG. 9 is a flow diagram showing a method for direct alkylamination of CNTs to form an Alk-CNT according to an embodiment of the present invention.

### DETAILED DESCRIPTION

In some aspects, the present disclosure provides methods of preparing aminated carbon nanotubes. The methods described herein may be used without the need for strong acid or at high temperature or pressure. These methods further comprise a solvent system that contains a high boiling point solvent such as a triol solvent, which may or may not further comprise water, such as deionized water.

### A. Methods Used Herein

The present methods may further comprise one or more carbon nanotubes. The carbon nanotubes (CNTs) used in various embodiments may be single walled, double walled or multi-walled CNTs. It is contemplated that these CNTs may be any CNT that is available. In some embodiments the CNTs have an average diameter in the range from about 1 nm to about 100 nm, from about 2 nm to about 75 nm, or from about 3 nm to about 50 nm. The average diameter of the CNTs is from about 1 nm, 2 nm, 3 nm, 5 nm, 10 nm, 20 nm, 30 nm, 40 nm, 50 nm, 60 nm, 70 nm, 80 nm, 90 nm, to about 100 nm, or any range derived therein.

In some embodiments the CNTs have an average length in the range from about 0.1 micron to about 150 microns, from about 0.5 microns to about 100 microns, or from about 1 micron to about 30 microns. The average length is from about 0.1 μm, 0.25 μm, 0.5 μm, 0.75 μm, 1 μm, 2.5 μm, 5 μm, 10 μm, 15 μm, 20 μm, 25 μm, 30 μm, 40 μm, 50 μm, 75 μm, 100 μm, 125 μm, to about 150 μm, or any range derivable therein. In other embodiments, the CNTs comprise at least one of reduced chemically CNTs. The CNTs may be as-synthesized or oxygen-free. In some embodiments, the mass percentage of carbon in the CNTs is in the range of 95.0%-99.9%. In some embodiments the CNT impurity component is Fe, Co, Ni, a metal oxide, such as metal oxide thereof, or combination thereof.

The method described herein may include the use of a deionized water-solvent mixture includes a triol-based solvent. The triol-based solvent may further comprise glycerol. In some embodiments, the deionized water-solvent mixture may include ethylene glycol, diethylene glycol, triethylene glycol, tetraethylene glycol, propylene glycol, any high boiling glycol, or a combination thereof. The triol-based solvent may further comprise one or more organic solvents such as an alcohol or be a mixture of multiple combinations of different glycerol or glycol. The solvent system may further comprise water especially deionized water. The deionized water content for the above mixtures may be in the range of 0% to 30% by volume, and preferably 0%-10% by volume. The amount of water may be from about 0%, 1%, 2%, 3%, 5%, 7%, 10%, 12.5%, 15%, 17.5%, 20%, 22.5%, 25%, 27.5%, to about 30%, or any range derivable therein.

Similarly, the present disclosure comprises using an aminating agent. The aminating agent may be urea. In other embodiments, the aminating agent is a chemical group comprising a function group of the formula: —NR′R″, wherein R′ or R″ are hydrogen, alkyl, alkenyl, or substituted versions of these groups. These substituted versions may include one or more non-hydrogen groups selected from the following list: —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Furthermore, the alkyl or alkenyl groups may each comprise from 1 to 30 carbon atoms. The number of carbon atoms may be from 6 to 18 carbon atoms. The number of carbon atoms may be 1, 2, 3, 4, 5, 6, 8, 10, 12, 14, 16, 18, 20, 22, 24, 26, 28, or 30 carbon atoms or any range derivable therein. Furthermore, the aminating agent may be further one or more trialkoxysilyl groups. Each of these alkoxy groups may be the same or different.

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkoxy” refers to the group —OR, in which R is an alkyl, as that term is defined above.

Furthermore, the present disclosure may comprise using a particular ratio of reagents to the CNT. The ratio of the CNT to any of the reagents is in the range of 0.1 to 5.0 by weight. This ratio of components may be from about 0.1, 0.25, 0.5, 1, 2, 3, 4, or 5 by weight. The methods can provide CNTs with high purity and low by-products. The methods result in a CNT that has a mass ratio of amine functional groups (—NH₂) to mass of CNT in the final ACNT powder is in the range of 0.001 to 0.02. The mass ratio of amine to mass of CNT is from about 0.001, 0.005, 0.001, or 0.0025, 0.005, 0.0075, 0.01, 0.015, to about 0.02. Similarly, the mass percentage of oxygen in the ACNT powder is in the range 0%<O %<0.5%. The mass percentage of oxygen may be less than about 0.025%, 0.05%, 0.075%, 0.1%, 0.15%, 0.2%, 0.25%, 0.3%, 0.35%, 0.4%, 0.45%, or 0.5%.

Similarly, the present methods contemplate using a specific temperature such as the temperature sufficient to reflux the solvents. The temperature may be from about 150° C. to about 250° C. The temperature may be from about 150° C., 160° C., 170° C., 180° C., 190° C., 200° C., 210° C., 220° C., 230° C., 240° C., to about 250° C., or any range derivable therein. In some aspects, the present methods require reacting the reaction mixture for a particular set of time. The fixed time of the reaction in the range from about 0.5 to about 10 hours or from about 1 hour to about 5 hours. The fixed time may be from about 0.5, 1, 2, 3, 4, 5, 6, 7, 8, 9, to about 10 hours, or any range derivable therein.

### II. EXAMPLES

### A. Facile Direct Amination Method of CNTs

Amination of CNTs is performed using urea with a high boiling point solvent under reflux. This allows for the synthesis to be easily scaled up, and for the reaction to be performed at 1 atmospheric pressure. Direct amination of the CNTs eliminates the need for pre-functionalization, and high-pressure reaction vessels. The use of a high boiling point solvent, like propylene glycol, makes it easy to synthesize using ambient pressure.

Experimental studies show successful amination of CNTs using a mixture of propylene glycol and urea under reflux at about 180° C. and 1 atmospheric pressure.

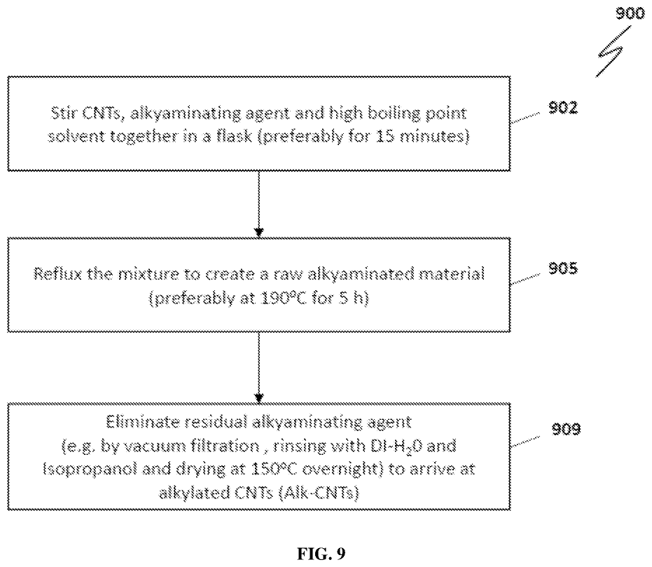

According to method 800 of FIG. 8, CNTs, an aminating agent and a solvent are mixed together to form a mixture as in step 802. In an alternate embodiment, a de-ionized water-solvent mixture is mixed with the CNTs and the aminating agent. The mixture is preferably stirred for about 15 minutes. In step 805, the mixture is reacted by reflux at a specific temperature and for a fixed time to create a raw aminated CNT compound. In step 809, residual aminating agent is eliminated, preferably by filtering, rinsing and drying, to produce the final aminated CNT (ACNT) powder.

In embodiments of method 800, the aminating agent may be urea, mono-alkyl amine or di-alkyl amine. For example, the aminating agent may include ethylenediamine, para-phenylenediamine, diethylamine, trimethylamine, hexylamine, octylamine, dodecylamine or a combination thereof. In some embodiments of method 800, urea is a preferable aminating agent. The ratio of the CNT to any of the reagents listed above is in the range of 0.1 to 5.0 by weight.

In some embodiments of method 800, the deionized water-solvent mixture includes a triol-based solvent comprising glycerol. In some embodiments of method 800, the deionized water-solvent mixture may include ethylene glycol, diethylene glycol, triethylene glycol, tetraethylene glycol, propylene glycol, any high boiling glycol, a triol based solvent, or a combination thereof. The deionized water content for the above mixtures may be in the range of 0% to 30% by volume, and preferably 0%-10% by volume.

According to embodiments of method 800, the specific temperature of the reflux reaction may be in the range of 150-250° C. and the fixed time of the reaction in the range of 1-10 hours. In some embodiments the fixed time of 1-5 hours is suitable.

According to embodiments of method 800, the mass ratio of amine functional groups (—NH₂) to mass of CNT in the final ACNT powder is in the range of 0.001 to 0.02 and wherein the mass percentage of oxygen in the ACNT powder is in the range 0%<O %<0.5%.

CNTs were obtained from Kumho Petrochemical. Acetone (ACS Reagent Grade), Isopropanol (IPA, 99.5%, ACS Reagent Grade), Urea (ACS Grade), Propylene Glycol (99%, ACS Reagent Grade) were purchased from Fisher Scientific. Ethanol (190 proof) was purchased from PHARMCO-AAPER. Potassium Bromide (KBr, FTIR grade) was purchased from Sigma Aldrich. Deionized water (DI-H₂O) was obtained from the lab. All chemicals were used as received, without any further purification. Functionalization of CNTs with —NH₂ groups was performed under reflux using urea as an amine source. In an embodiment of amination of graphene following the method 800, CNTs, urea, and propylene glycol were added to a 100 mL round bottom flask and stirred for 15 minutes. The mixture was then refluxed at 180° C. for 5 h, and let it cool to room temperature. ACNTs were then filtered, rinsed with acetone or isopropanol, and dried at 150° C. in order to eliminate any residual urea or ammonia present in the sample. After filtration, the filtrate was able to be re-used. Functionalization using the recycled solvent followed the same procedure as above.

Fourier transform infrared (FT-IR) spectroscopy was performed using Nicolet Avatar 360 FT-IR in order to confirm presence of functional groups. Thermal gravimetric analysis (TGA) was performed using a TA instruments Q600 simultaneous TGA/DSC in order to determine the thermal stability of the material. XPS analysis was performed using a PHI VersaProbe II instrument using Al-k alpha radiation as the x-ray source.

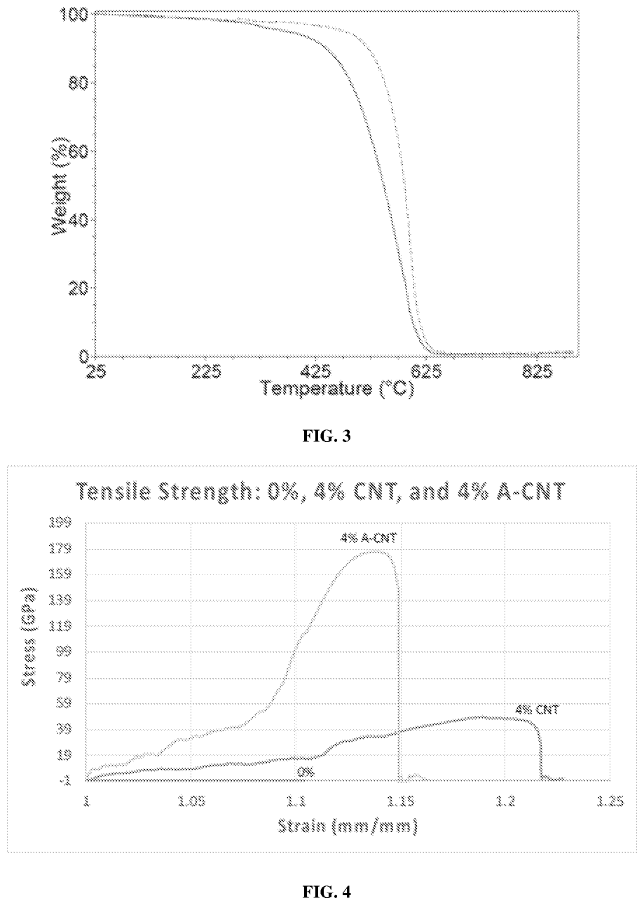

In order to confirm the presence of amine groups on the surface FT-IR analysis was performed on the material. FIG. 3 shows the analysis on the final material after washing and drying. Bands at 3500-3700 and ˜1623 cm⁻¹ correspond to the N—H bending and stretching respectively. While the band at ˜1020 cm⁻¹ corresponds to the aromatic C—N bonds.

To confirm amination X-ray photoelectron spectroscopy was performed on the material. FIG. 2 shows the N1s spectra for ACNTs, and the inset spectra is the N1s for pristine CNTs. Peaks for C—N and C—NH₂ were identified after peak fitting of the spectra which confirmed the presence of —NH₂ groups on the CNTs.

Thermal analysis was also performed on the amine functionalized CNTs and compared to the pristine CNTs to confirm that there was no presence of oxygen functionalities on the surface and confirm presence of amine functionalization. The TGA analysis (FIG. 3) shows that from 25° C.-300° C. the thermal degradation follows similar to graphene and then there is a sharp weight loss at approximately 300° C. corresponding to the loss of amine groups from the CNT surface (FIG. 3). This weight loss agrees with what has been reported in previous literature regarding thermal analysis of ACNT.

Prior work synthesizes aminated CNTs through the treatment of CNTs with concentrated acid followed by treated with an amine source. To confirm that no oxygen functionalities were present on the surface the TGA of ACNT was compared to that of pristine CNTs. Thermal analysis of ACNT shows weight loss occurring at approximately 440° C. corresponding to loss of amine groups from the surface. Where pristine CNTs do not show no significant weight loss until ˜585° C.

Instron measurements were taken on epoxy composites which included pristine CNTs and ACNTs. These measurements were performed on the ACNTs in order to observe its effects on mechanical properties of the epoxy resin. FIG. 4 shows the Instron graphs for pristine CNTs and ACNTs in epoxy composite.

Summarizing, a simple reflux method was discovered to synthesize aminated CNTs. The new method uses propylene glycol as solvent and urea as amination reagent to form aminated CNTs. This procedure also eliminates the need for treatment of pristine CNTs with concentrated acids, e.g., nitric acid, and caustic amines, such as ethylenediamine, used for the amination.

The aminated CNTs may be included as a component in an ink, a coating, a polymer, an engineering plastic, a rubber, a composite, a fiber, or a polymer film. Some embodiments of the present invention are realized as a coated surface, wherein the coated surface is a surface of an article having an applied coating comprising the amine functionalized CNTs resulting from the disclosed method for aminating CNTs. In some embodiments the coating may be a urethane coating, an epoxy coating or unsaturated polyester resin. In other embodiments, the composite may be an urethane carbon fiber composite, an urethane fiberglass composite, an epoxy carbon fiber composite, an epoxy fiberglass composite, an unsaturated polyester resin carbon fiber composite and an unsaturated polyester resin fiberglass composite. In yet other embodiments, the engineering plastic or polymer may be Nylon 6, Nylon 66, PET (polyester), PC (polycarbonate), other polar polymers or combinations thereof.

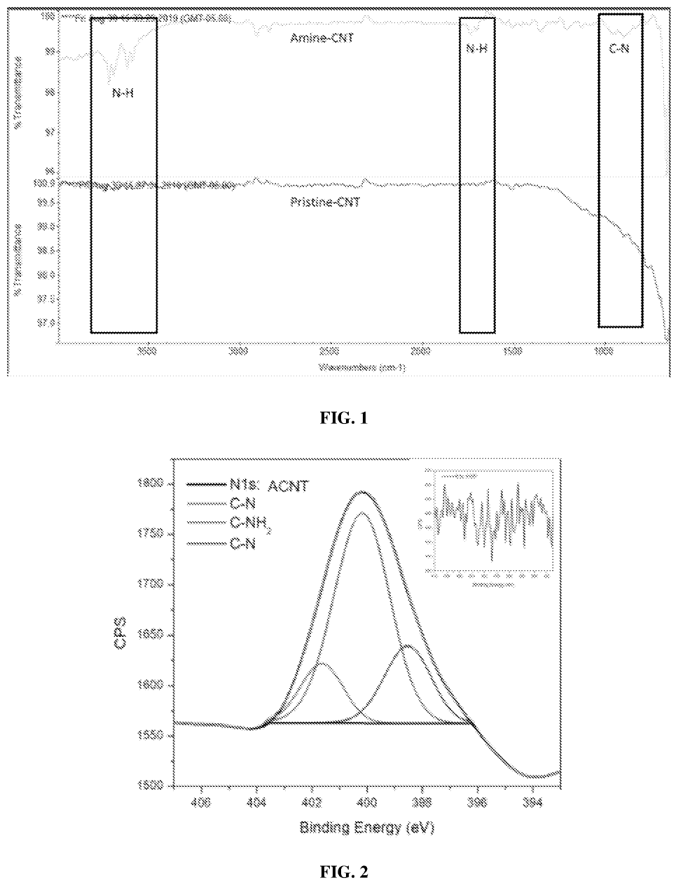

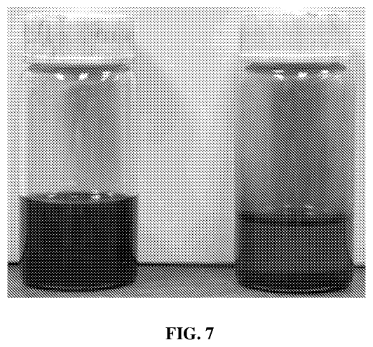

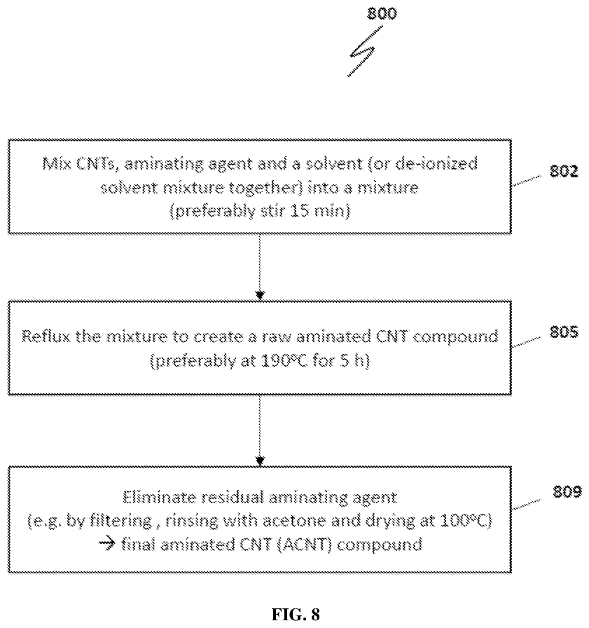

The illustration of the process for aminating graphene in the FIGS. 1-9 is not meant to imply physical or architectural limitations to the manner in which an illustrative embodiment may be implemented. Other components in addition to or in place of the ones illustrated may be used. Some components may be unnecessary. Also, the blocks are presented to illustrate some functional components. One or more of these blocks may be combined, divided, or combined and divided into different blocks when implemented in an illustrative embodiment.

### B. Facile Direct Alkylamination Method of CNTs

Alkylaminated CNTs can have enhanced mechanical properties so it can give benefits as an additive in polymers or engineering plastics. In the direct alkylamination process disclosed herein is performed using an alkylaminating agent (C₇-C₂₀) with high boiling point solvent under reflux as described in method 900 of FIG. 9.

According to method 900, CNTs, an alkylaminating agent and a high boiling point solvent are mixed together to form a mixture as in step 902. The mixture is preferably stirred for about 15 minutes. In step 905, the mixture is reacted by reflux at a specific temperature and for a fixed time to create a raw alkylaminated CNT compound. In step 909, residual alkylaminating agent is eliminated, preferably by vacuum filtration, rinsing and drying, to produce the final alkylaminated CNT (Alk-CNT) powder.

In embodiments of method 900, the alkylaminating agent may be mono-alkyl amine or di-alkyl amine. For example, the alkylaminating agent may be hexylamine (C6), octylamine (C8), dodecylamine (C12), oleylamine (C18) or any alkylamine with Cn where n>6. The ratio of the CNT to any of the reagents listed above is in the range of 0.1 to 5.0 by weight.

In some embodiments of method 900, the deionized water-solvent mixture includes a triol-based solvent comprising glycerol. In some embodiments of method 800, the deionized water-solvent mixture may include ethylene glycol, diethylene glycol, triethylene glycol, tetraethylene glycol, propylene glycol, any high boiling glycol, a triol based solvent, or a combination thereof. The deionized water content for the above mixtures may be in the range of 0% to 30% by volume, and preferably 0%-10% by volume.

According to embodiments of method 900, the specific temperature of the reflux reaction may be in the range of 150-250° C. and the fixed time of the reaction in the range of 1-10 hours. In some embodiments the fixed time of 1-5 hours is suitable.

According to embodiments of method 900, the mass ratio of alkylamine group to CNT in the final Alk-CNT powder is in the range of 0.005 to 0.05 and wherein the mass percentage of oxygen in the Alk-CNT powder is in the range 0%<O %<0.5%.

Direct alkylamination of CNTs was conducted using a reflux method, under atmospheric pressure, using alkylaminating agents with various chain lengths, and high boiling point solvent. In an embodiment of direct alkylamination of CNTs following the method 900, the direct alkylamination of CNTs is performed in a round bottom flask, under reflux, at atmospheric pressure with a high boiling point solvent and varying alkylaminating agents. CNTs are placed inside the round bottom flask, followed by addition of solvent and alkylaminating agent. The flask is heated to 190° C., reflux is started, and reaction is held for 5 h. Reaction is cooled to room temperature then raw alkylaminated CNTs (Alk-CNTs) are collected. Alk-CNTs are rinsed with water and isopropanol, then dried to ensure all un-reacted alkylaminating agent and solvent is gone.

For example, Ig of CNTs are placed inside a 100 mL round bottom flask followed by the addition of alkylaminating agent, hexylamine, and high boiling point solvent, propylene glycol. This was stirred for 15 minutes to ensure homogenous dispersion. After stirring the round bottom flask was lowered to an oil bath, followed by attachment of condenser on the flask. The hotplate was then turned on an allowed to reach a temperature of 190° C., then the water was turned on for refluxing. The reaction was carried out for 5 h. Alkylaminated raw material was collected using vacuum filtration, followed by rinsing with DI-H₂O and isopropanol, then drying in a vacuum oven at 150° C. overnight to eliminate excess alkylaminating agent and solvent.

Alk-CNTs were characterized by FTIR and thermogravimetric analysis (TGA) to ensure the incorporation of alkyl groups on the surface and ends of the CNTs.

Fourier transform infrared (FT-IR) spectroscopy was performed using Nicolet Avatar 360 FT-IR in order to confirm presence of functional groups. Thermal gravimetric analysis (TGA) was performed using a TA instruments Q600 simultaneous TGA/DSC in order to determine the thermal stability of the material and to confirm that there was no presence of oxygen groups in the sample.

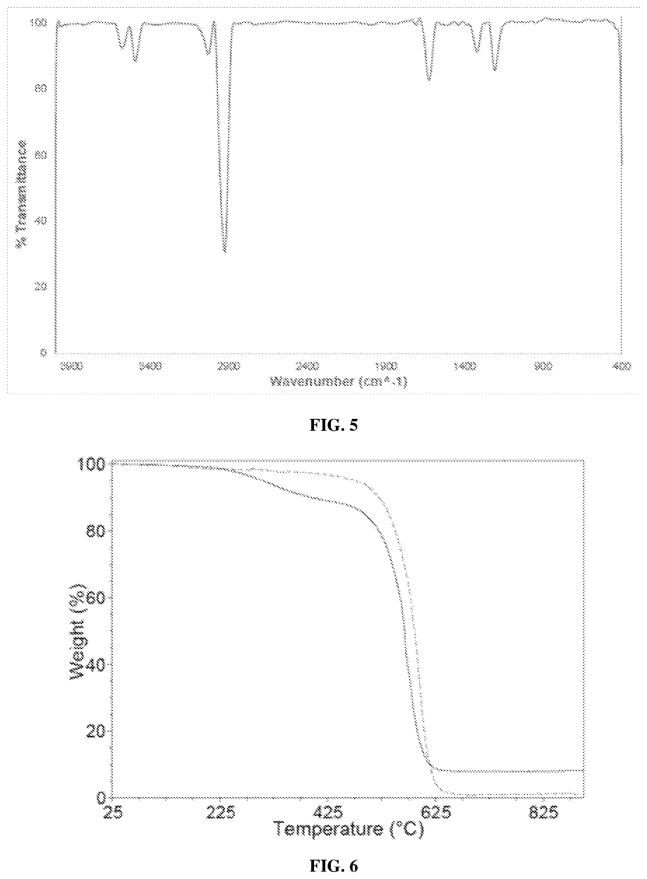

FTIR spectrum of alkylaminated CNTs (Alk-CNTs) are shown (FIG. 5) in which alkylaminated CNTs shows —C—H and —C—N peaks.

FIG. 6 shows a thermal degradation profile of pristine CNTs vs. alkylaminated CNTs. Alkylamination is evident by the degradation observed at 245° C. compared to the pristine CNTs samples. The small weight loss observed at approximately 245° C. corresponds to the loss of alkyl groups from the Alk-CNT.

Dispersion studies were done on Alk-CNT material in non-polar solvent systems. These showed that presence of alkyl groups on the CNTs helped to enhance dispersion stability by 2 weeks versus pristine CNTs which crashed out immediately.

In summary, direct alkylamination of CNTs according to method 900 successfully produces alkylaminated CNTs (Alk-CNTs). Chemical characterization confirmed the presence of alkyl groups in CNTs. In some embodiments, the Alk-CNTs may be included as a component in an ink, a coating, a polymer, an engineering plastic, a rubber, a composite, a fiber, or a polymer film. Addition of Alk-CNTs into an engineering plastic or composite coating can help to achieve enhanced physical properties, such as mechanical or thermal, resulting from addition of Alk-CNT. Engineering plastics containing Alk-CNTs may be polypropylene, polyethylene, TPO (thermoplastic poly olefins), rubber, other non-polar plastics and combinations thereof. Some embodiments of the present invention are realized as a coated surface, wherein the coated surface is a surface of an article having an applied coating comprising the alkyl functionalized CNTs resulting from the process for alkylaminating CNTs.

The illustration of the processes for aminating and alkylaminating CNT in the FIGS. 1-9 is not meant to imply physical or architectural limitations to the manner in which an illustrative embodiment may be implemented. Other components in addition to or in place of the ones illustrated may be used. Some components may be unnecessary. Also, the blocks are presented to illustrate some functional components. One or more of these blocks may be combined, divided, or combined and divided into different blocks when implemented in an illustrative embodiment.

The terminology used herein was chosen to best explain the principles of the embodiment, the practical application or technical improvement over technologies found in the marketplace, or to enable others of ordinary skill in the art to understand the embodiments disclosed here. All the compositions and methods disclosed and claimed herein can be made and executed without undue experimentation in light of the present disclosure. The descriptions of the various embodiments of the present invention were presented for purposes of illustration but are not intended to be exhaustive or limited to the embodiments disclosed. For example, variations may be applied to the compositions and methods and in the steps or in the sequence of steps of the methods described herein. More specifically, it will be apparent that certain agents which are both chemically and physiologically related, may be substituted for the agents described herein while the same or similar results would be achieved. All such similar substitutes and modifications apparent to those skilled in the art are deemed to be within the spirit, scope and concept of the disclosure as defined by the appended claims.

### III. REFERENCES

The following references, to the extent that they provide exemplary procedural or other details supplementary to those set forth herein, are specifically incorporated herein by reference.
Abdelkader Fernández et al., Catalysis Science & Technology, 7 (15), 3361-3374 (2017).
Alam et al., Polym Composite, 35 (11), 2129-2136 (2014).
Barreiro et al., The Journal of Physical Chemistry, 110 (42), 20973-20977 (2006).
Basiuk et al., Nano Letters, 4 (5), 863-866 (2004).
Basiuk et al., J of Nanoscience and Nanotechnology, Vol. X. 1-7, (2005).
Dresselhaus et al., Philos Trans A Math Phys Eng Sci, 366 (1863), 231-6 (2008).
Ferreira et al., Applied Surface Science, 410, 267-277 (2017).
Raja et al., Soft Mater, 6 (2), 65-74 (2008).
Shanmugharaj et al., Compos Sci Technol, 67 (9), 1813-1822 (2007).
Zhang et al., Applied Surface Science, 343, 19-27 (2015).

1. A method for preparing an aminated carbon nanotube (CNT), comprising reacting a CNT nanomaterial with an aminating reagent in a triol based solvent or a triol based solvent-deionized water mixture to obtain the aminated CNT, wherein the CNT nanomaterial comprises a mass percentage of carbon in the CNT nanomaterial in the range of 95.0%-99.9%.

2. The method of claim 1, wherein the method is substantially free of any acid or oxidant.

3.-6. (canceled)

7. The method of claim 1, wherein the CNT has an average diameter in the range of 3 nm to 50 nm or an average length in the range of 1 μm to 30 μm.

8.-10. (canceled)

11. The method of claim 1, wherein the aminating reagent is selected from the group consisting of urea, monoalkyl amine, dialkyl amine, monoalkenyl amine, or dialkenyl amine.

12.-13. (canceled)

14. The method of claim 1, wherein the aminating reagent is urea.

15. The method of claim 1, wherein the ratio of CNT to aminating reagent is in the range of 0.1-5.0 by weight.

16. (canceled)

17. The method of claim 1, wherein the deionized watersolvent mixture includes ethylene glycol, diethylene glycol, triethylene glycol, tetraethylene glycol, propylene glycol, any high boiling glycol, or a combination thereof.

18. The method of claim 1, wherein the deionized watersolvent mixture has a deionized water content in the range of 0%-30% by volume.

19. The method of claim 1, wherein the temperature of the reaction is in the range of 150-250° C. at about 1 atmospheric pressure.

20. (canceled)

21. The method of claim 1, wherein the reaction is carried out under reflux in air.

22.-24. (canceled)

25. A method for preparing an alkylaminated CNT comprising reacting a CNT nanomaterial with an alkylaminating reagent in a triol based solvent or a triol based solvent-deionized water mixture to obtain the alkylaminated CNT, wherein the CNT comprises a mass percentage of carbon in the CNT nanomaterial in the range of 95.0%-99.9%.

26.-28. (canceled)

29. The method according to claim 25, wherein the CNT has an average diameter in the range of 3 nm to 50 nm or an average length in the range of 1 μm to 30 μm.

30.-32. (canceled)

33. The method of claim 25, wherein the alkylaminating reagent is a monoalkyl amine, monoalkenyl amine, dialkyl amine or dialkenyl amine.

34. (canceled)

35. The method of claim 25, wherein the alkylaminating reagent is an alkyl or alkenyl group of C5 to C20 hydrocarbons.

36.-39. (canceled)

40. The method of claim 25, wherein the ratio of CNT to alkylaminating reagent is in the range of 0.1-5.0 by weight.

41. (canceled)

42. The method of claim 25, wherein the deionized water-solvent mixture includes ethylene glycol, diethylene glycol, triethylene glycol, tetraethylene glycol, propylene glycol, any high boiling glycol, or a combination thereof.

43.-44. (canceled)

45. The method of claim 25, wherein the temperature of the reaction is in the range of 150-250° C. at about 1 atmospheric pressure.

46. (canceled)

47. The method of claim 25, wherein the reaction is carried out under reflux in air.

48.-50. (canceled)

51. An aminated CNT, obtained in a method of claim 1, wherein the mass ratio of amine functional groups to mass of CNT is in the range of 0.001 to 0.05.

52. (canceled)

53. An alkylaminated CNT, obtained in a method of claim 25, wherein the mass ratio of alkylamine group to CNT is in the range of 0.005-0.05.

54.-61. (canceled)
